(19) United States
(12) Patent Application Publication
(10) Pub. No.: US 2025/0340576 A1
(43) Pub. Date: Nov. 6, 2025

(54) IMPROVED PROCESS FOR PREPARING METAL ALKOXIDE COMPOUNDS

(71) Applicant: Evonik Operations GmbH, Essen (DE)

(72) Inventors: Niklas PAUL, Marl (DE); Moritz SCHRÖDER, Münster (DE); Armin Matthias RIX, Marl (DE); Dirk ROETTGER, Köln (DE); Philip ZITZEWITZ, Haltern am See (DE); Johannes RUWWE, Hanau (DE)

(73) Assignee: Evonik Operations GmbH, Essen (DE)

(21) Appl. No.: 19/137,401

(22) PCT Filed: Dec. 1, 2023
(86) PCT No.: PCT/EP2023/083872

§ 371 Date: Jun. 10, 2025

(30) Foreign Application Priority Data
Dec. 14, 2022 (WO) PCT/EP2022/085748

(51) Int. Cl. C07F 1/04

(52) U.S. Cl. CPC C07F 1/04

(57) ABSTRACT

The present invention relates to a process for preparing metal alkoxide compounds MOR² from the metal hydroxides MOH and the compounds of the formulae R¹OH and R²OH, where the boiling point of R¹OH is lower than that of R²OH. R¹ and R² here are alkyl radicals or haloalkyl radicals, the carbon chain of which may be interrupted by ether groups, and which may have hydroxy groups. M here is a metal, preferably an alkali metal.

The process, by contrast with the conventional processes for transalcoholization, which require at least two reaction steps in two different reactive distillation columns, is conducted as a multiple reactive distillation in a reactive distillation column. This results in a decrease in apparatus complexity and a reduction in the need for power and heating steam. The process is especially suitable for preparation of compounds MOR² for which the corresponding compound R²OH forms an azeotrope with water and/or for which the boiling point of R²OH is close to the boiling point of water.

The present invention relates to a process for preparing metal alkoxide compounds MOR² from the metal hydroxides MOH and the compounds of the formulae R¹OH and R²OH, where the boiling point of R¹OH is lower than that of R²OH. R¹ and R² here are alkyl radicals or haloalkyl radicals, the carbon chain of which may be interrupted by ether groups, and which may have hydroxy groups. M here is a metal, preferably an alkali metal.

The process, by contrast with the conventional processes for transalcoholization, which require at least two reaction steps in two different reactive distillation columns, is conducted as a multiple reactive distillation in a reactive distillation column. This results in a decrease in apparatus complexity and a reduction in the need for power and heating steam. The process is especially suitable for preparation of compounds MOR² for which the corresponding compound R²OH forms an azeotrope with water and/or for which the boiling point of R²OH is close to the boiling point of water.

### BACKGROUND OF THE INVENTION

Alkali metal alkoxides are used as strong bases in the synthesis of numerous chemicals, for example in the production of pharmaceutical or agrochemical active ingredients. Alkali metal alkoxides are also used as catalysts in transesterification and amidation reactions.

Alkali metal alkoxides are prepared by electrolysis, for example, as described in EP 3 885 470 A1.

Alkali metal alkoxides (MOR) are additionally also prepared by reactive distillation according to reaction <A> below in a countercurrent distillation column from alkali metal hydroxides (MOH) and alcohols (ROH, e.g. methanol), wherein the water of reaction formed is removed with the distillate.

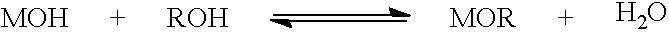

This “conventional” process principle (i.e. the production of alkali metal alkoxides from alkali metal hydroxides and the corresponding alcohol ROH by reactive distillation) is described, for example, in GB 737 453 A, U.S. Pat. Nos. 4,566,947 A, 2,877,274 A, EP 0 091 425 A2, DD 246 988 A1, WO 01/42178 A1, CN 109 627 145 A, CN 208632416 U, WO 2021/148174 A1 and WO 2021/148175 A1. This involves conducting aqueous alkali metal hydroxide solution and gaseous alcohol (for example methanol, ethanol, propanol or butanol) in countercurrent in at least one reactive distillation column. The most industrially important alkali metal alkoxides here are those of sodium and potassium, and here especially the methoxides and ethoxides. Their synthesis is frequently described in the prior art, for example in EP 1 997 794 A1.

Methods that are similar, but in which an entraining agent, for example benzene, is additionally used, are described in GB 377,631 A and U.S. Pat. No. 1,910,331 A. This entraining agent is used to separate water and the water-soluble alcohol. In both patent specifications the condensate is subjected to a phase separation to separate off the water of reaction.

Correspondingly, DE 96 89 03 C describes a method of continuous preparation of alkali metal alkoxides in a reaction column, wherein the water-alcohol mixture withdrawn at the top of the column is condensed and then subjected to a phase separation. The aqueous phase is discarded and the alcoholic phase is returned to the top of the column together with the fresh alcohol. EP 0 299 577 A2 describes a similar method, wherein the water in the condensate is separated off with the aid of a membrane.

The syntheses of the alkali metal alkoxides from alkali metal hydroxides and the corresponding alcohol ROH by reactive distillation as described in the prior art typically afford vapours comprising the alcohol used and water. It is advantageous for economic reasons to reuse the alcohol present in the vapours as a reactant in the reactive distillation. The vapours are therefore typically fed to a rectification column and the alcohol present therein is separated off (described for example in EP 4 074 684 A1, EP 4 074 685 A1, WO 2021/148174 A1 and WO 2021/148175 A1). The alcohol thus recovered is then fed to the reactive distillation as a reactant.

In this conventional method according to reaction <A>, the alcohol ROH of which the alkoxide MOR is to be prepared is accordingly typically recovered in the vapour from the reaction column as a mixture with water. This is disadvantageous under some circumstances, for example when the boiling point of the alcohol of the alkoxide prepared is close to that of water, since in that case the vapour can be separated by distillation only with a high level of complexity.

A particular difficulty also arises in the case of alcohols that form azeotropes with water. Vapours that are obtained in the above-described “conventional” reactive distillation according to <A> and comprise water and alcohols that form azeotropes with water (for example ethanol) can then be separated into their constituents by distillation only with great difficulty. The preparation of alkali metal alkoxides is of ethanol, but also n-propanol or iso-propanol, via this “conventional route” from alkali metal hydroxide solution and the respective alcohol thus harbours disadvantages. This lack of flexibility in the process regime in the “conventional” reactive distillation, the reason for which is that the composition of the vapour depends on the alkoxide prepared and the vapour includes not only water but typically also the corresponding alcohol, is a disadvantage of these processes. It is desirable to be able to adjust the composition of the vapour “flexibly”, i.e. independently of the alkoxide prepared.

In order to avoid this problem, it is possible to use a similar process for preparing metal alkoxides (“MOR”). This alternative process is based on the reaction according to scheme <B>:

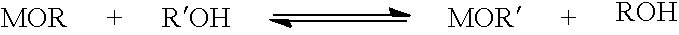

This is the reaction of an alkali metal alkoxide MOR with an alcohol R′OH other than ROH. This “transalcoholization” is described, for example, in CS 213119 B1 and is advantageously conducted in a reaction column, as described in WO 2021/122702 A1, U.S. Pat. No. 3,418,383 A and DE 27 26 491 A1.

Typically, R′OH is an alcohol having a higher boiling point than ROH; for example, ROH=methanol, and R′OH is an alcohol having a longer alkyl chain (R′ is, for example, ethyl, n-propyl, iso-propyl or a butyl isomer).

The conventional processes for preparing metal alkoxides by transalcoholization accordingly comprise two stages proceeding from metal hydroxide MOH and the two alcohols R′OH and ROH:

In a first step I, the first metal alkoxide MOR is prepared from metal hydroxide MOH and a first alcohol ROH.

MOR is then reacted in the subsequent step II with a further alcohol R′OH to give MOR′ and ROH.

This way of preparing alkali metal alkoxides MOR′ permits greater flexibility in the process regime than the above-described reaction <A> and is an option particularly in the cases in which R′OH is an alcohol that forms azeotropes with water or the boiling points of R′OH and H₂O are close to one another. If, for example, higher alkoxides (i.e. those wherein the alkyl radical is heavier than methyl) are prepared by transalcoholization from metal methoxide, what are typically obtained (in step I) are solely aqueous vapours that additionally comprise methanol, and have good distillative separability as methanol/water mixtures.

Nevertheless, disadvantages arise here too: If the reactions are conducted by reactive distillation, two reactive distillation columns are needed: one for step I and one for step II. Since two reaction columns have to be operated, this means high apparatus complexity and entails a high energy demand.

It was therefore an object of the present invention to provide a process for preparing metal alkoxides and similar compounds that does not have the aforementioned disadvantages and is notable in particular for lower apparatus complexity and minimized energy demand.

### BRIEF DESCRIPTION OF THE INVENTION

A process which achieves the object of the invention has now surprisingly been found.

The present invention accordingly relates to a process for preparing a compound of the formula MOR², wherein
(a) a reactant stream S₁ comprising a compound of the formula R¹OH is fed via a lateral feed into a reactive distillation column RR optionally having a bottoms circuit S_{U1}, and with a column section B above the feed point and a column section A below the feed point,
(b) a reactant stream S₀ comprising a compound of the formula MOH is fed into column section B,
(c) reactant stream S₁ is reacted with reactant stream S₀ in countercurrent in column section B to give a crude product RP_{B} comprising MOR¹, water and R¹OH, with or without MOH,
(d) a reactant stream S₂ comprising a compound of the formula R²OH is fed into column section A,
(e) reactant stream S₂ is reacted with the compound MOR¹ obtained in step (c) in countercurrent in column section A to give a crude product RPA comprising MOR², R²OH and R¹OH, with or without MOR¹,
(f) a bottom product stream S_{U} comprising MOR² and R²OH is withdrawn from the bottom of RR and, if column RR has a bottoms circuit S_{U1}, alternatively or additionally from the bottoms circuit S_{U1} of RR,
(g) and a vapour stream S₀ comprising water and R¹OH is withdrawn at the upper end of RR,
where M is a metal,
and where R¹ is an alkyl radical optionally having one or more hydroxy groups, or a haloalkyl radical optionally having one or more hydroxy groups,
and where R² is an alkyl radical optionally having one or more hydroxy groups, or a haloalkyl radical optionally having one or more hydroxy groups,
where, for R¹, the carbon chain of the alkyl radical or haloalkyl radical may be interrupted by one or more oxygen atoms, where there are at least two carbon atoms between interrupting oxygen atoms and any hydroxy group included in R¹,
where, for R², the carbon chain of the alkyl radical or haloalkyl radical may be interrupted by one or more oxygen atoms, where there are at least two carbon atoms between interrupting oxygen atoms and any hydroxy group included in R²,
and where R¹ and R² are different.

### FIGURES

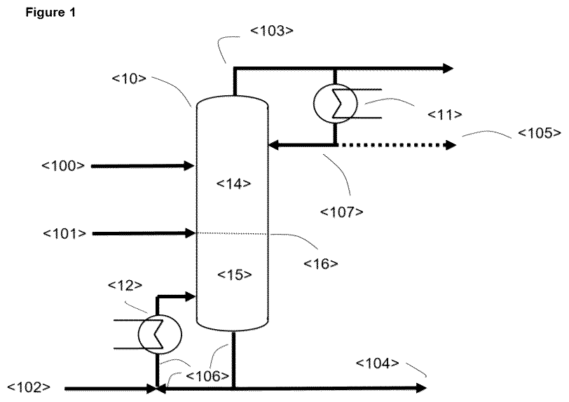

### FIG. 1

FIG. 1 shows one embodiment of the process according to the invention.

In this process, a stream S₁ of gaseous methanol <101> is directed into a rectification column RR <10>. In the rectification column RR <10>, two reactions are conducted in the upper column section B <14> and in the lower column section A <15> (“multiple reactive distillation”).

A stream S₀ <100> of a 50% by weight NaOH solution is directed into column section B <14> above the feed for stream S₁ <101>. In column section B <14>, the two streams S₀ <100> and S₁ <101> are reacted with one another in countercurrent, forming a crude product RP_{B} comprising sodium methoxide. Sodium methoxide accumulates in column section A <15>, which adjoins column section B <14> (the boundary <16> between A <15> and B <14> is indicated schematically by the dotted line). In addition, there may be devices mounted between column sections A and B in order to separate these from one another and to better prevent the aqueous vapour in B from coming into contact with compound R²OH in A and forming an azeotrope. Such an apparatus may, for example, be a tray (sieve tray, bubble-cap tray, valve tray).

In column section A <15>, NaOCH₃ reacts with ethanol to give sodium ethoxide. Ethanol is fed as liquid stream S₂ <102> via the bottoms circuit S_{U1} <106> into column section A <15>. S₂ <102> is introduced into the bottoms circuit S_{U1} <106> before being guided through an evaporator <12>, which is especially a forced circulation evaporator. Sodium ethoxide is withdrawn from the bottom of the column RR <10> as a solution in ethanol and ultimately from the bottoms circuit S_{U1} <106> as bottom stream S_{U} <104>.

At the top of the column RR <10>, a vapour stream S_{O} <103> comprising methanol and water is withdrawn, which is partly condensed in a heat exchanger <11>, and the condensate is applied as reflux <107> to the column <10> and may be partly discharged from the process in liquid form as stream <105>. The portion of the vapour stream S_{O} <103> which is not refluxed via <11> is fed to the rectification column for separation of methanol and water (not shown in FIG. 1).

Alternatively, it is also possible to condense the entire vapour stream S_{O} <103> in <11>, in which case at least a portion is recycled to column <10>, while the other portion <105> is discharged from the process in liquid form and optionally separated, for example by distillation.

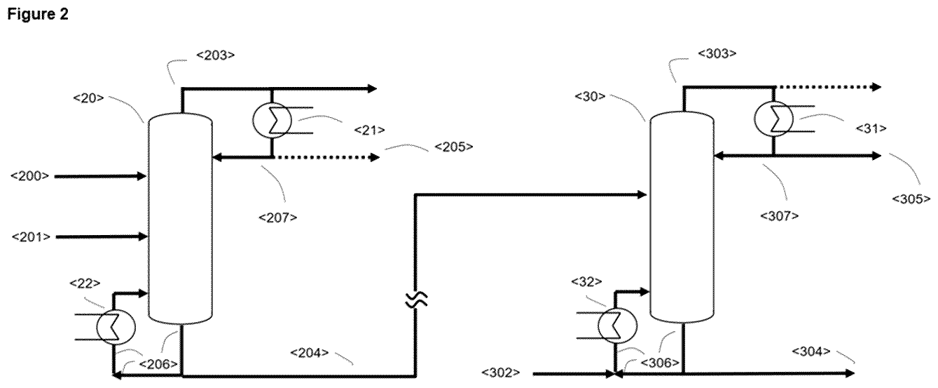

### FIG. 2

FIG. 2 shows a noninventive comparative process. This comprises two reaction columns <20> and <30> corresponding to the aforementioned column RR <10>.

A stream of gaseous methanol <201> and a stream of a 50% by weight NaOH solution <200> are directed into the reaction column <20>. The two streams <200> and <201> are reacted with one another in countercurrent, forming a crude product comprising sodium methoxide which accumulates in the bottom of the column <20>. At the bottom of the column <20>, a methanolic solution of sodium methoxide is withdrawn and recycled into the bottom of the column <20> via an evaporator <22> as bottoms circulation stream <206>. The bottom product stream <204> is discharged from the bottoms circuit.

At the top of the column <20>, a vapour stream <203> comprising methanol and water is withdrawn, which is partly condensed in a condenser <21>, and this portion is applied as reflux <207> to the column <20> and a portion of the condensate is optionally discharged from the process in liquid form as <205>. The portion of the vapour stream <203> which is not run via <21> is fed to a rectification column for separation of methanol and water (not shown in FIG. 2). Alternatively, it is also possible to condense the entire vapour stream <203> in <21>, in which case at least a portion <207> is recycled to column <20>, while the other portion <205> is discharged from the process in liquid form.

The methanolic sodium methoxide solution <204> is fed to a further rectification column <30>. The transalcoholization takes place in the reaction column <30>. It has a bottoms circuit <306> which is run through an evaporator <32>. Liquid ethanol <302> is fed into the bottoms circuit <32>. This is directed into the column <30> via the evaporator <32> and reacted in the column <30> with stream <204> to give a crude product comprising methanol, ethanol and sodium ethoxide. A bottom stream comprising an ethanolic solution of sodium ethoxide <304> is withdrawn from the bottoms circuit <32> and discharged from the process.

At the top of the column <30>, a vapour stream <303> comprising methanol and water is withdrawn, which is partly or fully condensed in the condenser <31>, and the condensate is partly applied as reflux <307> to the column <30> and is optionally discharged from the process in liquid form as stream <305>. The uncondensed portion of the vapour stream <303> is fed to a rectification column for separation of methanol and water (not shown in FIG. 2). This column may be the same rectification column in which the uncondensed portion of stream <203> is also separated. It is thus possible to separate portions of both streams <203> and <303> in one rectification column in order to save energy.

Alternatively, it is also possible to condense the entire vapour stream SO <303> in <31>, in which case at least a portion <307> is recycled to column <30>, while the other portion <305> is discharged from the process in liquid form.

Compared to the process shown in FIG. 1, the process according to FIG. 2 is more complex in terms of apparatus, since two columns <20> and <30> are needed to conduct a reaction sequence comparable to that of the process according to the invention.

### DETAILED DESCRIPTION OF THE INVENTION

The present invention relates to a process for preparing a compound of the formula MOR² by reactive distillation.

The process according to the invention is based on two reactions.

In a first reaction, according to the following reaction <C1>, the compound MOR¹ is obtained:

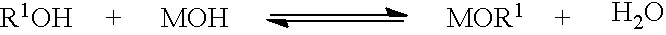

Compound MOR¹ is then reacted in the subsequent reaction <C2>, corresponding to a transalcoholization, with compound R²OH to give compound MOR²:

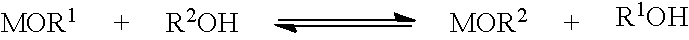

R¹ is an alkyl radical or haloalkyl radical that optionally has one or more hydroxy groups, where, for R¹, the carbon chain of the alkyl radical or haloalkyl radical may be interrupted by one or more oxygen atoms, where there are at least two carbon atoms between interrupting oxygen atoms and any hydroxy group included in R¹.

R² is an alkyl radical or haloalkyl radical that optionally has one or more hydroxy groups, where, for R², the carbon chain of the alkyl radical or haloalkyl radical may be interrupted by one or more oxygen atoms, where there are at least two carbon atoms between interrupting oxygen atoms and any hydroxy group included in R².

R¹ and R² are different. The person skilled in the art will also appreciate that the compound R²OH will have a higher boiling point than R¹OH since this is a necessary prerequisite for R¹OH to be obtained at the top and R²OH at the bottom of column RR. This prerequisite is met automatically when R¹=methyl.

The compound of the formula MOR² is referred to in the context of the invention as “metal alkoxide compound”. “Metal alkoxide compound” in the context of the invention is especially understood to mean metal alkoxides and metal ether alkoxides. According to the invention, the metal alkoxide compound is preferably a metal alkoxide.

When the compound MOR² is a metal ether alkoxide, R² is an alkyl radical optionally having one or more hydroxy groups, and optionally interrupted by one or more oxygen atoms, where there are at least two carbon atoms between interrupting oxygen atoms and any hydroxy group encompassed by R².

An oxygen atom interrupting an alkyl radical, where there are at least two carbon atoms between the latter and any further oxygen atoms interrupting the alkyl radical and any hydroxy group encompassed by the alkyl radical, is referred to as “ether group”.

When the compound MOR² is a metal alkoxide, R² is an alkyl radical optionally having one or more hydroxy groups.

“Alkyl” in the context of the invention includes “cycloalkyl”.

“Haloalkyl” is preferably an alkyl radical in which at least one hydrogen atom has been exchanged for a halogen atom, where the halogen atom is more preferably selected from fluorine, chlorine.

In a preferred embodiment, R¹ is alkyl, more preferably C₁ to C₄-alkyl, even more preferably methyl or ethyl, and most preferably methyl.

More preferably, R¹ is methyl, and R² is selected from the group consisting of C₂ to C₁₀-alkyl, —(CH₂)₂OH, —(CH₂)₂O(CH₂)₂OH, —(CH₂)₃OH, —(CH₂)₄OH, 1-methoxypropan-2-yl.

Yet more preferably, R¹ is methyl, and R² is C₂ to C₁₀-alkyl. Yet more preferably, R¹ is methyl, and R² is selected from the group consisting of ethyl, iso-propyl, sec-butyl, 2-methyl-2-butyl, tert-butyl, 2-methyl-2-pentyl, 3-methyl-3-pentyl, 3-ethyl-3-pentyl, 2-methyl-2-hexyl, 3-methyl-3-hexyl.

Yet more preferably, R¹ is methyl, and R² is selected from the group consisting of ethyl, iso-propyl, sec-butyl, 2-methyl-2-butyl, 3-methyl-3-pentyl, 3-ethyl-3-pentyl.

Yet more preferably, R¹ is methyl, and R² is selected from the group consisting of ethyl, iso-propyl, 2-methyl-2-butyl.

Most preferably, R¹=methyl and R²=ethyl.

In another preferred embodiment, R¹=methyl and R²OH is a compound that forms an azeotropic mixture with water. These especially include ethanol, n-propanol, iso-propanol, n-butanol, sec-butanol, iso-butanol, tert-butanol, cyclohexanol, preferably ethanol, n-propanol, iso-propanol. Most preferably, the compound R²OH that forms an azeotropic mixture with water is ethanol.

In a further preferred embodiment, the boiling point of compound R²OH at standard pressure (1 bar) is in the range from 70° C. to 130° C., preferably in the range of 78° C. to 120° C.

M is a metal, especially an alkali metal, preferably selected from lithium, sodium, potassium, more preferably from sodium, potassium.

Most preferably, M=sodium.

The process according to the invention can be performed either continuously or batchwise. The process according to the invention is preferably performed continuously. Within the reactive distillation column RR, constant mass transfer and the changing concentrations within the gas phase and the liquid phase result in constant readjustment of the reaction equilibrium, which enables a high conversion.

Preferably, reactant streams S₀, S₁, S₂ are fed simultaneously into reactive distillation column RR.

The process according to the invention is conducted especially at a temperature within a range from 45° C. to 150° C., preferably within a range from 47° C. to 120° C., more preferably within a range from 60° C. to 110° C., and at a pressure within a range from 0.2 bar abs. to 40 bar abs., preferably 0.5 bar abs. to 10 bar abs., preferably within a range from 0.7 bar abs. to 5 bar abs., more preferably within a range from 0.8 bar abs. to 4 bar abs., more preferably within a range from 0.9 bar abs. to 3.5 bar abs., yet more preferably within a range from 1.0 bar abs. to 3 bar abs., yet more preferably at 1.25 bar abs.

The equilibrium position of the reactions underlying the process according to the invention is temperature-dependent in the preparation of some compounds MOR², especially the alkoxides. In these cases, high temperatures may advantageously result in a higher conversion. It may also be advantageous to conduct the process according to the invention under elevated pressure, for example at least 1.5 bar absolute, at least 2.5 bar absolute, or at least 5.0 bar absolute.

In other embodiments, it is advantageous to conduct the process according to the invention under reduced pressure, for example within a range of 0.1 to 0.9 bar abs., especially 0.3 to 0.75 bar abs.

### Step (a)

In step (a) of the process according to the invention, a reactant stream S₁ comprising a compound of the formula R¹OH is fed via a lateral feed into a reactive distillation column RR optionally having a bottoms circuit S_{U1}, and with a column section B above the feed point and a column section A below the feed point.

The word “lateral” is understood to mean that the feed is below the top of the column and above the bottom of the reactive distillation column RR. The feed point of stream S₁ divides the column into a column section B (above the feed point) and a column section A (below the feed point).

The reaction corresponding to the aforementioned reaction <C1> takes place in the reaction column RR above the feed point for stream S₁ (step (c) of the process according to the invention). The reaction corresponding to the aforementioned reaction <C2> takes place in the reaction column RR below the feed point for stream S₁ (step (e) of the process according to the invention). If the stream S₁ is directed into the reaction column RR via multiple feed points, column section B is above the feed point closest to the top of the column RR, and column section A is below the feed point closest to the top of the column RR.

According to the invention, “reactive distillation column” (synonym: “reactive rectification column”) defines a distillation column in which the reaction according to the invention as per the above reaction equations <C1> and <C2> proceeds at least in some parts. It can also be abbreviated to “reaction column” or, in the context of the present invention, to “column”.

According to the invention, column section A (as reaction section for reaction <C2>) and column section B (as reaction section for reaction <C1>) are arranged one on top of another in a single column RR. Since two reactions thus proceed in column RR in the present process, column RR may also be referred to as a “multiple reaction column”, and the process according to the invention as a “multiple reactive distillation”.

The reactive distillation column RR used may be a standard reactive distillation column. Column RR is selected, for example, from columns with random packing, columns with structured packing and tray columns, more preferably tray columns and columns with structured packing.

Installed in suitable tray columns are sieve trays, bubble-cap trays or valve trays, through which the liquid phase flows. The reactive distillation column in the process according to the invention preferably has trays as internals, for example selected from bubble-cap trays, valve trays, bubble-cap trays, cross-slit bubble-cap trays, sieve trays, Thormann® trays.

Columns with random packing may be filled with different random packings. Heat and mass transfer are improved by the increase in the surface area on account of the shaped bodies that are usually about 25 to 80 mm in size. Examples are the Raschig ring (a hollow cylinder), Pall ring, Hiflow ring, Intalox saddle and the like. The random packings may be introduced into the column in an ordered manner or else randomly (as a bed). Useful materials include glass, ceramic, metal and plastics.

Structured packings are a further development of the ordered random packings. They have a regular-shaped structure. It is thus possible in the case of structured packings to reduce pressure drops in the flow of gas. There are various designs of structured packings, for example fabric packings or sheet metal packings. Column section B preferably comprises structured packings, while column section A comprises trays.

The top of the column refers to the region free of internals above the uppermost tray, or above the uppermost layer of structured packing. It is generally formed by a curved plate (hood, e.g. dished end or torispherical head), which forms the concluding element of the reactive distillation column. According to the invention, the top of the column RR is also part of column section B.

The bottom of the column refers to the region free of internals below the lowermost tray, or below the lowermost layer of structured packing. According to the invention, the bottom of column RR is part of column section A. According to the invention, if column RR has a bottoms circuit S_{U1}, the bottoms circuit S_{U1} is also part of column section A.

The suitable number of theoretical plates in column RR depends on the difference in the vapour pressures of R¹OH and R²OH, with a greater number of theoretical plates being advantageous in the case of a smaller difference. It also depends on the equilibrium position of reactions <C1> and <C2>, where the advantage of a greater number of theoretical plates increases with the extent to which the equilibrium is to the reactant side. The suitable number of theoretical plates in the two column sections also depends on the purity of bottom product and top product which is to be achieved, and—if a reflux is established—the reflux rate used, where a higher number of theoretical plates is required to achieve a higher purity for a given reflux rate.

The reactant stream S₁ comprises a compound of the formula R¹OH. In a preferred embodiment, the proportion by mass of all compounds of the formula R¹OH in S₁ is >95% by weight, yet more preferably >99% by weight, and S₁ otherwise comprises especially water.

The reactant stream S₁ preferably comprises methanol. In a more preferred embodiment, the proportion by mass of methanol in S₁ is then >95% by weight, yet more preferably ≥99% by weight, and S₁ otherwise comprises especially water.

The methanol used in step (a) as reactant stream S₁ in this preferred embodiment may also be commercially available methanol having a proportion by mass of methanol of more than 99.8% by weight and a proportion by mass of water of up to 0.2% by weight.

The reactant stream S₁ is preferably introduced into step (a) in vapour form.

### Step (b)

In step (b) of the process according to the invention, a reactant stream S₀ comprising a compound of the formula MOH is fed into column section B.

The reactant stream S₀ comprises MOH. In a preferred embodiment, especially when R¹OH=methanol, S₀ comprises not only MOH but also at least one further compound selected from water, methanol. It is yet more preferable when S₀ also comprises water in addition to MOH; in that case, S₀ is an aqueous solution of MOH.

When the reactant stream S₀ comprises MOH and water, the proportion by mass of MOH, based on the total weight of the aqueous solution forming S₀, is especially within a range from 10% to 75% by weight, preferably in the range from 15% to 54% by weight, more preferably in the range from 30% to 53% by weight and especially preferably in the range from 40% to 52% by weight.

When the reactant stream S₀ comprises MOH and methanol, the proportion by mass of MOH in methanol, based on the total weight of the solution forming S₀, is especially within a range from 10% to 75% by weight, preferably in the range from 15% to 54% by weight, more preferably in the range from 30% to 53% by weight, and especially preferably in the range from 40% to 52% by weight.

In the particular case in which the reactant stream S₀ comprises both water and methanol in addition to MOH, it is particularly preferable that the proportion by mass of MOH in methanol and water, based on the total weight of the solution forming S₀, is especially within a range from 10% to 75% by weight, preferably in the range from 15% to 54% by weight, more preferably in the range from 30% to 53% by weight, and especially preferably in the range from 40% to 52% by weight.

### Step (c)

In step (c) of the process according to the invention, reactant stream S₁ is reacted with reactant stream S₀ in countercurrent in column section B to give a crude product RP_{B} comprising MOR¹, water and R¹OH, with or without MOH.

In step (c), the reaction accordingly takes place according to the aforementioned reaction equation <C1>.

The “reaction of the reactant stream S₁ comprising a compound of the formula R¹OH with reactant stream S₀ comprising a compound of the formula MOH in countercurrent” is achieved in accordance with the invention in that the feed point for reactant stream S₁ in step (a) in reaction column RR is below the feed point for reactant stream S₀ in step (b). In particular, S₁ is added in vaporous form, and S₀ as a solution, such that the two streams S₀ and S₁ meet and are reacted with one another in column section B.

The reaction column RR preferably comprises at least 1, in particular at least 2, preferably 15 to 40, theoretical plates between the feed point of the reactant stream S₁ and the feed point of the reactant stream S₀.

In column section B of the reaction column RR, the reactant stream S₁ comprising a compound of the formula R¹OH is then reacted with the reactant stream S₀ comprising a compound of the formula MOH in the above-described reaction <C1> to give MOR¹ and H₂O, where these products, since the reaction is an equilibrium reaction, are present in a mixture with the reactant R¹OH and possibly (since R¹OH is especially added in molar excess to MOH) the reactant MOH. Accordingly, a crude product RP_{B} comprising not only the products MOR¹ and water but also R¹OH and possibly MOH is obtained in step (c) in column section B of the reaction column RR.

At the same time, the more volatile components become enriched in the gas phase in the direction of the top of column RR, and the less volatile components in the liquid phase in the direction of the bottom of the column RR.

A portion of compound R¹OH is thus in gaseous form in the reactive distillation column RR and ascends as vapour in the direction of the top of column RR. As a result, a vapour comprising R¹OH is obtained in column section B. This is drawn off at the top of column RR in step (d) of the process according to the invention as vapour stream S_{O} comprising R¹OH. At the same time, at the upper end of column section B of RR (and hence at the upper end of RR), water is also driven out as a vapour together with R¹OH and withdrawn in step (g) as vapour stream S_{O}. Even if the boiling point of compound R¹OH is below the boiling point of water, the feed of reactant stream S₁ can be adjusted such that the water of reaction and the water added with stream S₀ are driven out as a vapour.

At the lower end of column section B of RR, compound MOR¹ is then obtained, which is converted further in column section A in step (e) and in accordance with the above reaction equation <C2>.

In a preferred embodiment of the process according to the invention, and especially in the cases in which S₀ comprises not only MOH but also water, the ratio of the total weight (mass; unit: kg) of all compounds R¹OH that are used as reactant stream S₁ in step (a) to the total weight (mass; unit: kg) of all compounds MOH used as reactant stream S₀ in step (b) is in the range from 4:1 to 50:1, more preferably in the range from 8:1 to 48:1, even more preferably in the range from 10:1 to 45:1, more preferably in the range from 20:1 to 40:1, even more preferably 22:1.

### Step (d)

In step (d) of the process according to the invention, a reactant stream S₂ comprising a compound of the formula R²OH is fed into column section A.

“Feeding reactant stream S₂ into column section A” in step (d) comprises feeding below the feed point of reactant stream S₁, especially into the bottom of column RR, and, if column RR has a bottoms circuit S_{U1}, alternatively or additionally into the bottoms circuit S_{U1} of column RR. Feeding into column section A is effected in the cases in which RR has a bottoms circuit S_{U1}, preferably in such a way that S₂ is fed into the bottoms circuit S_{U1}.

Stream S₂ in step (d) can be fed in liquid form or gaseous form into column section A, especially the bottom, or in the cases in which RR has a bottoms circuit S_{U1}, alternatively or additionally into the bottoms circuit S_{U1} of column RR. Preferably, stream S₂ in step (d) is fed in liquid form into column section A, especially the bottom, or in the cases in which RR has a bottoms circuit S_{U1}, alternatively or additionally into the bottoms circuit S_{U1} of column RR.

More preferably, column RR has a bottoms circuit S_{U1} into which stream S₂ is fed in liquid form in step (d).

“Bottoms circuit S_{U1}” is understood to mean the portion of the bottom stream withdrawn from column RR which is fed back into column RR. In FIG. 1, the bottoms circuit S_{U1} is thus formed by stream <106>. This recycled portion of the bottom stream (in FIG. 1: <106>) is preferably heated by means of a forced circulation evaporator (in FIG. 1: <12>) before being fed back into column RR.

The feeding of S₂ into the bottoms circuit S_{U1} can accordingly be effected into the bottom stream before stream S_{U} (in FIG. 1: <104>) is withdrawn from the bottoms circuit S_{U1} (in FIG. 1: <106>).

Alternatively, S₂ can be fed into the bottoms circuit S_{U1} after stream S_{U} has been removed from the bottoms circuit Sur. If a forced circulation evaporator is used, S₂ can also be fed directly into the forced circulation evaporator.

In a preferred embodiment of the process according to the invention, the molar ratio of the molar amount (unit: mol) of all compounds R¹OH that are used as reactant stream S₁ in step (a) to the molar amount (unit: mol) of all compounds R²OH used as reactant stream S₂ in step (d) is in the range from 9:1 to 1:9, more preferably in the range from 8:1 to 1:2, even more preferably in the range from 7:1 to 1:1, more preferably in the range from 5:1 to 2:1, even more preferably 4.3:1.

### Step (e)

In step (e) of the process according to the invention, stream S₂ which is fed in in step (d) and the compound MOR¹ which is obtained in step (c), in the context of the crude product RP_{B}, are reacted with one another in countercurrent in column section A. This affords a crude product RPA comprising MOR², R²OH, R¹OH and possibly MOR¹.

In step (e), the reaction accordingly takes place according to the aforementioned reaction equation <C2>.

As already described similarly in connection with step (c), the constituents of the crude product RPA will accumulate in column section A according to their volatility. The more volatile components such as R¹OH accumulate in the gas phase in the direction of the top of column RR, while the less volatile components MOR² and R²OH accumulate in the liquid phase in the direction of the bottom of the column.

At least a portion of the compound R¹OH obtained as crude product RPA is thus in gaseous form in column section A of the reactive distillation column RR after step (e) has been performed, and rises in the form of vapour in the direction of the top of column RR, where it mixes with the R¹OH in the crude product RP_{B} in column section B, and the vapour accumulates in column section B. This vapour is drawn off at the top of column RR in step (g) of the process according to the invention as vapour stream S_{O} comprising R¹OH and water.

At least a portion of the compounds MOR², R²OH obtained as crude product RPA is thus in liquid form in column section A of the reactive distillation column RR and accumulates in the bottom of column RR after performance of step (e). In step (f) of the process according to the invention, it is then withdrawn from the bottom of RR as a bottom product stream S_{U} comprising MOR² and R²OH, and, if column RR has a bottoms circuit S_{U1}, alternatively or additionally from the bottoms circuit S_{U1} of RR.

### Step (f)

In step (f) of the process according to the invention, a bottom product stream S_{U} comprising MOR² and R²OH is withdrawn from the bottom of RR and, if column RR has a bottoms circuit S_{U1}, alternatively or additionally from the bottoms circuit S_{U1} of RR.

“A bottom product stream S_{U} comprising MOR² and R²OH is withdrawn from the bottom of RR and, if column RR has a bottoms circuit S_{U1}, alternatively or additionally from the bottoms circuit of RR” means in accordance with the invention that, in the cases in which column RR has a bottoms circuit S_{U1}, the bottom product stream S_{U}, alternatively or additionally to direct withdrawal from the bottom, can be withdrawn from the bottoms circuit S_{U1}. This is the case, for example, in an embodiment in which a bottom product stream comprising MOR² and R²OH is withdrawn from the bottom of column RR, then this is recycled into column RR as bottoms circulation stream S_{U1}, optionally via a forced circulation evaporator, and S_{U} is withdrawn from the bottoms circuit S_{U1} as bottom product stream S_{U} (for example discharged from the process).

The stream S_{U} drawn off at the bottom of the column RR and/or from the bottoms circuit S_{U1} typically consists essentially of R²OH and the product MOR². Stream S_{U} can therefore be used further as it is, optionally after cooling in a heat exchanger, or else stored.

It is optionally possible to separate R²OH from MOR² in stream S_{U} in order to increase the concentration of MOR². Alternatively, further R²OH may be added to stream S_{U} in order to decrease the concentration of MOR² in stream S_{U}.

The solution of MOR² in R²OH drawn off as stream S_{U} advantageously includes only a small amount of compound R¹OH, which permits an efficient process. Preferably, the proportion of all compounds R¹OH in the solution drawn off as stream S_{U} is not more than 1.0% by weight, preferably not more than 0.5% by weight, more preferably not more than 0.3% by weight, even more preferably <0.01% by weight, yet more preferably <0.001% by weight, for example 0.001% to 0.20% by weight or 0.01% to 0.10% by weight, based on the total weight of the solution drawn off as stream S_{U}.

The solution of MOR² in R²OH drawn off as stream S_{U} likewise advantageously includes only small amounts of water, which permits an efficient process. Preferably, the proportion of water in the solution drawn off as stream S_{U} is not more than 1.0% by weight, preferably not more than 0.5% by weight, more preferably not more than 0.3% by weight, even more preferably <0.01% by weight, yet more preferably <0.001% by weight, for example 0.001% to 0.20% by weight or 0.01% to 0.10% by weight, based on the total weight of the solution drawn off as stream S_{U}.

If R¹=methyl, the methanol concentration in the solution can be determined, for example, by headspace analysis or by gas chromatography, as described in WO 2021/122702 A1.

The proportion of all compounds of the formula MOR² in the solution drawn off as stream S_{U} is especially in the range of 3% to 60% by weight, preferably 5% to 55% by weight and more preferably 7% to 50% by weight, for example 7% to 30% by weight, 15% to 25% by weight, 19% to 25% by weight or 21% to 24% by weight, based on the total weight of the solution drawn off as stream S_{U}.

According to the concentration of compounds MOR² and R²OH in stream S_{U}, stream S₂ can also be used to dilute stream S_{U}. Then stream S₂ is fed into the bottom stream in the bottoms circuit S_{U1} of the column in step (d) before bottom stream S_{U} (labelled “<104>” in FIG. 1) is withdrawn from the bottoms circuit S_{U1} (labelled “<106>” in FIG. 1).

The concentration of the compounds of the formula MOR² in the solution drawn off as stream S_{U} can be determined, for example, by titration, as described in WO 2021/122702 A1.

The reactive distillation column RR typically has an evaporator, preferably a forced circulation evaporator. This evaporator may be integrated in the column bottom. But it is preferably an evaporator accommodated in the bottoms circuit S_{U} (=“circulation evaporator”). In this case, in particular, a substream of the stream drawn off at the bottom of the column can be fed to the bottoms circuit S_{U1} and then returned to the column as a heated, possibly biphasic fluid stream.

Alternatively or additionally, the bottoms are heated directly.

Suitable evaporators are, for example, boilers, natural-circulation evaporators, forced circulation evaporators and forced circulation flash evaporators.

In the case of forced circulation evaporators, a pump is used to conduct the liquid to be evaporated through the heater. The resultant vapour/liquid mixture is then returned to the column RR.

In the case of forced circulation flash evaporators, which is a particular embodiment of forced circulation evaporators, a pump is likewise used to conduct the liquid to be evaporated through the heater. A superheated liquid recycle stream is obtained, which is expanded into the bottom of the column. The pressure on the solution drawn off from column RR, which is returned to the column, is increased by superheating. The superheated recycle stream is expanded through a flow limiter. This results in superheating of the liquid above its boiling point in relation to the pressure within the column.

On passage of the superheated liquid through the flow limiter and reentry into the column, the liquid is evaporated abruptly. This abrupt evaporation proceeds with a considerable increase in volume and leads to acceleration of the fluid flow entering the column. Advantageously, the flow limiter is disposed immediately upstream of the reentry of the superheated liquid into the column, or even within it. The flow limiter used is preferably a diaphragm, a valve, a throttle, a perforated plate, a nozzle, a capillary or combinations thereof, especially a valve. For example, it is possible to use a rotary plug valve. It is particularly preferable when the opening characteristics of the flow limiter are adjustable. In this way, it is possible always to keep the pressure in the evaporator above the boiling pressure of the liquid, based on the pressure within the column, even in the case of changed flow rates, as can occur, for example, in startup and shutdown operations. It is advantageous that operating the evaporator by forced circulation or by forced circulation flashing achieves an elevated flow rate of the liquid in the heating apparatus compared to operation with natural circulation, for example in the tube bundle of the heat exchanger. The elevated flow rate results in improved heat transfer between heat exchanger and heated liquid, which in turn contributes to avoidance of local superheating.

The pump to be used in the case of forced circulation or forced circulation flash evaporators is preferably disposed between the withdrawal conduit and the evaporator.

In a preferred embodiment of the process according to the invention, the reactive distillation column RR has a forced circulation evaporator and stream S₂ is fed in liquid form into the feed to the forced circulation evaporator.

Alternatively or additionally to the forced circulation evaporator, the bottoms may be heated directly, for example by means of a boiler.

The bottom temperature of the reactive distillation column RR at a given pressure determines the concentration of the compound MOR² in the solution drawn off as stream S_{U} at the bottom of column RR or from the bottoms circuit S_{U1}. The temperature and hence the concentration are appropriately chosen such that compound MOR² always remains in solution in the bottoms. The bottom temperature is adjusted, for example, by means of an evaporator and/or direct heating of the bottoms.

In a further embodiment, the reactive distillation column RR is filled with R²OH prior to startup, and R²OH is at first also used as reflux. On attainment of the operating temperature, streams S₀ and S₁ are then fed in.

### Step (g)

In step (g) of the process according to the invention, a vapour stream S₀ comprising water and R¹OH is withdrawn at the upper end of RR.

This vapour stream S₀ comprising water and R¹OH is preferably directed at least partly into a rectification column RD_{A}, where it is separated by distillation at least partly into water and R¹OH. According to the boiling point of R¹OH relative to the boiling point of H₂O, water or R¹OH is withdrawn at the bottom or top of RD_{A}.

If R¹OH=methanol, the separation in RD_{A} is into at least one vapour stream S_{OA} comprising R¹OH which is withdrawn at the upper end of RD_{A}, and at least one stream S_{UA} comprising water which is withdrawn at the lower end of RD_{A}.

At least a portion of the R¹OH, especially methanol, obtained in the distillation in RD_{A} can be used as reactant stream S₁ in step (a).

When R¹=methyl, it is accordingly preferable that at least a portion of vapour stream S₀ is directed into a rectification column RD_{A} and is separated in RD_{A} into at least one vapour stream S_{OA} comprising methanol which is withdrawn at the upper end of RD_{A}, and at least one stream S_{UA} comprising water which is withdrawn at the lower end of RD_{A}.

Even more preferably, in that case, at least a portion of stream S_{OA} is used as reactant stream S₁ in step (a).

The reaction column RR is operated with or without, preferably with, reflux.

“With reflux” means that the vapour stream S₀ withdrawn at the upper end of the respective column, in step (g) that withdrawn from the reaction column RR, is not conducted away completely. In step (g), the vapour stream S₀ in question is then fed at least partly, preferably partly, back to the reaction column RR as reflux. In the cases where such a reflux is established, the reflux ratio is preferably 0.01 to 1, more preferably 0.02 to 0.9, yet more preferably 0.03 to 0.34, yet more preferably 0.04 to 0.27, yet more preferably 0.05 to 0.24, yet more preferably 0.06 to 0.10, yet more preferably 0.07 to 0.09. Generally and in the context of the present invention, a reflux ratio is understood to mean the ratio of the proportion of the mass flow withdrawn from the column (kg/h) that is recycled into the column in liquid form (reflux) to the proportion of this mass flow (kg/h) that is discharged from the respective column in liquid form or gaseous form.

A reflux can be established by mounting a condenser at the top of the respective column. A condenser K_{RR} may be mounted, for example, atop the reaction column RR. In the condenser K_{RR}, the vapour stream S₀ is condensed at least partly, preferably partly, and the condensate is fed back to the reaction column RR.

In the embodiment in which a reflux is established in the reaction column RR, the MOH used as reactant stream S₀ in step (b) may also be at least partly mixed with the reflux stream, and the resulting mixture may be supplied as such to the reaction column RR.

### Advantages

The process according to the invention accordingly permits the advantages described for transalcoholization (flexibility in the process regime, which is important particularly in the case of alcohols having a similar boiling point to water, or alcohols that form azeotropes with water). Compared to the prior art transalcoholization processes, there is additionally a distinct saving of energy and minimization of apparatus complexity.

### EXAMPLE

### Example 1 (Inventive)

In the apparatus according to FIG. 1, a gaseous methanol stream S₁ <101 > of 5500 kg/h is run into a multiple reactive distillation column RR <10>. In the upper section B <14> of column RR <10>, a 50% sodium hydroxide solution S₀ <100> of 550 kg/h is run in countercurrent.

In the upper section B <14> of column RR <10>, NaOH and methanol are converted to sodium methoxide in the first forty trays.

In the lower section A <15> of column RR <10>, the transalcoholization of sodium methoxide to sodium ethoxide is effected. For this purpose, 1800 kg/h of S₂ <102 > ethanol is run into the bottoms circuit S_{U1} <106> of the multiple reactive distillation column RR <10>. In the lower section A <15> of column RR <10>, the transalcoholization takes place.

At the bottom of column RR, 1900 kg/h of sodium ethoxide (solution in ethanol) is separated off, which is recycled as bottoms circulation stream S_{U1} <106> into column RR <10>, and stream S_{U} <104> is led off from the bottoms circuit S_{U1<106>.}

A 99% methanol stream S₀<103> is drawn off overhead, which can be fed partly to a further rectification column RD_{A} for workup and partly recycled to column RR <10> as reflux <107>.

### Example 2 (Non-Inventive)

The amount of sodium ethoxide corresponding to Example 1 is prepared according to the prior art as outlined in FIG. 2, i.e. methanolic sodium methoxide solution is first obtained (step I) from sodium hydroxide solution and methanol in column <20> (cf. Example 2.3 of EP 1 997 794 A1). Thereafter (step II), this methanolic solution of NaOCH₃ is reacted with ethanol in a further reaction column <30> in a transalcoholization to give ethanolic sodium ethoxide solution (as described in WO 2021/122702 A1).

### Example 3 (Non-Inventive)

Ethanolic sodium ethoxide solution is obtained from aqueous sodium hydroxide solution and ethanol according to Example 2.3 of EP 1 997 794 A1, using the corresponding amount of ethanol (1035 g) rather than the amount of 720 g of methanol (22.5 mol) specified therein.

### Results

The advantages of the process according to the invention are directly apparent from comparison of Example 1 with the conventional methods shown in Examples 2 and 3:

1. In the comparative process according to Example 2, for preparation of ethoxide by transalcoholization from the corresponding methoxide (step II), the prior preparation of the methoxide from methanol and aqueous sodium hydroxide solution (step I) is necessary. This entails the operation of two reactive distillation columns <20> and <30>. By contrast, in the process according to the invention, only one reactive distillation column <10> is required. Thus, the apparatus complexity and the heating output to be provided are halved.

2. In non-inventive Example 3, only one reaction column is used, but this is not a transalcoholization. This has the above-described disadvantages of the “conventional” process regime. Thus, in the preparation of corresponding ethoxides, vapours are obtained in which water is present in a mixture with ethanol. Ethanol, which forms azeotropes with water, can then be separated from the water in the vapour only in a complex manner. This problem is observed for all alkoxides for which the alcohols form azeotropes with water (for example including iso-propanol and n-propanol) or which have boiling points close to those of water.

By contrast, in the case of the procedure according to the invention, it is possible to avoid such azeotropic mixtures, since the alcohol present in a mixture with water in the vapour is different regardless of the alcohol for which the alkoxide is obtained. In the reaction column RR in Example 1, contact of ethanol and water is minimized, since ethanol is formed in the lower section A <15> and water in the upper section B <14> of the reaction column RR, and they are largely separated from one another by virtue of the vaporous methanol fed in as stream S₁ <101>. Thus, the process according to the invention permits preparation of sodium ethoxide by transalcoholization from sodium methoxide. It thus provides the advantages of a transalcoholization, but without having the disadvantages thereof with regard to high energy demand.

1-13. (canceled)

14. A process for preparing a compound of formula MOR², wherein:
(a) a reactant stream S₁ comprising a compound of formula R¹OH, is fed via a lateral feed into a reactive distillation column RR at a feed point, and wherein RR optionally has a bottoms circuit S_{U1}, a column section B above the feed point and a column section A below the feed point;
(b) a reactant stream S₀ comprising a compound of formula MOH is fed into column section B;
(c) reactant stream S₁ is reacted with reactant stream S₀ in countercurrent in column section B to give a crude product RP_{B} comprising MOR¹, water, and R¹OH;
(d) a reactant stream S₂ comprising a compound of the formula R²OH is fed into column section A;
(e) reactant stream S₂ is reacted with the compound MOR¹ obtained in step (c) in countercurrent in column section A to give a crude product RPA comprising MOR², R²OH, and R¹OH;
(f) a bottom product stream S_{U} comprising MOR² and R²OH is withdrawn from the bottom of RR and, if column RR has a bottoms circuit S_{U1}, alternatively or additionally from the bottoms circuit S_{U1} of RR; and
(g) a vapour stream S₀ comprising water and R¹OH is withdrawn at the upper end of RR;
wherein:
M is a metal;
R¹ is an alkyl radical optionally having one or more hydroxy groups, or a haloalkyl radical optionally having one or more hydroxy groups,
R² is an alkyl radical optionally having one or more hydroxy groups, or a haloalkyl radical optionally having one or more hydroxy groups,
and wherein,
for R¹ and R², the carbon chain of the alkyl radical or haloalkyl radical may be interrupted by one or more oxygen atoms, wherein there are at least two carbon atoms between interrupting oxygen atoms and any hydroxy group included in R¹ or R², and
R¹ and R² are different.

15. The process of claim 14, wherein reactant streams S₀, S₁, S₂ are fed simultaneously into reactive distillation column RR.

16. The process of claim 14, wherein M is an alkali metal.

17. The process of claim 14, wherein R¹ is methyl.

18. The process of claim 17, wherein R²OH is a compound that forms an azeotropic mixture with water.

19. The process of claim 17, wherein R² is selected from the group consisting of C₂ to C₁₀-alkyl, —(CH₂)₂OH, —(CH₂)₂O(CH₂)₂OH, —(CH₂)₃OH, —(CH₂)₄OH, and 1-methoxypropan-2-yl.

20. The process of claim 19, wherein R² is selected from the group consisting of ethyl, iso-propyl, sec-butyl, 2-methyl-2-butyl, tert-butyl, 2-methyl-2-pentyl, 3-methyl-3-pentyl, 3-ethyl-3-pentyl, 2-methyl-2-hexyl, and 3-methyl-3-hexyl.

21. The process of claim 17, wherein at least a portion of vapour stream S_{O} is directed into a rectification column RD_{A} and is separated in RD_{A} into at least one vapour stream S_{OA} comprising methanol which is withdrawn at the upper end of RD_{A}, and at least one stream S_{UA} comprising water which is withdrawn at the lower end of RD_{A}.

22. The process of claim 21, wherein at least a portion of stream S_{OA} is used as reactant stream S₁ in step (a).

23. The process of claim 14, wherein stream S₂ in step (d) is fed in liquid form into column section A.

24. The process of claim 14, wherein stream S₂ in step (d) is fed into the bottom of column RR, and, if column RR has a bottoms circuit S_{U1}, alternatively or additionally, stream S₂ in step (d) is fed into the bottoms circuit S_{U1} of column RR.

25. The process of claim 24, wherein column RR has a bottoms circuit S_{U1}.

26. The process of claim 25, wherein the bottoms circuit S_{U1} of column RR comprises a forced circulation evaporator, and stream S₂ is fed in liquid form into the feed to the forced circulation evaporator.

27. The process of claim 15, wherein R¹ is methyl.

28. The process of claim 27, wherein R²OH is a compound that forms an azeotropic mixture with water.

29. The process of claim 27, wherein R² is selected from the group consisting of C₂ to C₁₀-alkyl, —(CH₂)₂OH, —(CH₂)₂O(CH₂)₂OH, —(CH₂)₃OH, —(CH₂)₄OH, and 1-methoxypropan-2-yl.

30. The process of claim 29, wherein R² is selected from the group consisting of ethyl, iso-propyl, sec-butyl, 2-methyl-2-butyl, tert-butyl, 2-methyl-2-pentyl, 3-methyl-3-pentyl, 3-ethyl-3-pentyl, 2-methyl-2-hexyl, and 3-methyl-3-hexyl.

31. The process of claim 30, wherein at least a portion of vapour stream S_{O} is directed into a rectification column RD_{A} and is separated in RD_{A} into at least one vapour stream S_{OA} comprising methanol which is withdrawn at the upper end of RD_{A}, and at least one stream S_{UA} comprising water which is withdrawn at the lower end of RD_{A}.

32. The process of claim 31, wherein at least a portion of stream S_{OA} is used as reactant stream S₁ in step (a).

33. The process of claim 32, wherein stream S₂ in step (d) is fed in liquid form into column section A.
